(19) United States
(12) Patent Application Publication
(10) Pub. No.: US 2004/0158195 A1
(43) Pub. Date: Aug. 12, 2004

(54) Needle-free mass injection device

(76) Inventors: Gary J. Sibert, Lebanon, CT (US); Paul J. Dominowski, Old Lyme, CT (US); Joseph C. Frantz, East Lyme, CT (US)

Correspondence Address: SCULLY SCOTT MURPHY & PRESSER, PC, 400 GARDEN CITY PLAZA, GARDEN CITY, NY, 11530

(21) Appl. No.: 10/359,394

(22) Filed: Feb. 6, 2003

(51) Int. Cl. A61M 5/30

(52) U.S. Cl. 604/68

(57) ABSTRACT

A needle-fee injection device including: a housing having a striker channel, a dose chamber, and a jet orifice; a syringe plunger movable in the dose chamber between reload and ejection positions; a striker movable within the striker channel between the reload and ejection positions; a spring for storing energy to be delivered to the striker; a driven member; an engagement member movable between engagement and disengagement positions with the driven member such that engagement of the engagement member with the driven member causes the spring to store energy and disengagement of the engagement member with the driven member allows the stored energy in the spring to be delivered to the striker causing the striker to impact the syringe plunger and eject the medicament from the dose chamber through the jet orifice.

### BACKGROUND OF THE INVENTION

1. Field of the Invention

The present invention relates generally to delivery of medicaments without the use of a needle, more particularly, a multiple-use needle-free injection device that utilizes high fluid pressure to penetrate the skin and pass medicaments into or through the skin.

2. Prior Art

In human and veterinary medicine, it is known to administer medicaments through the use of a needle and syringe or through the use of other instruments generally referred to as needle-free injectors. Prior art needle-free injector devices employ a mechanical compression spring, compressed inert gas, or an internal combustion engine to create sufficient energy transfer via a plunger to propel fluid through a small orifice. The amount of energy directed against the plunger provides sufficient pressure in the nozzle to accelerate fluid medicaments to an average velocity between 400 feet per second and 1000 feet per second. An initial velocity spike of greater than 1000 feet per second is known to be ideal to create a channel in the skin that will then permit deposition of the entire dose, but at velocities less than 1000 feet per second. It is also known that the volume of medicament and delivery pressure will determine the depth of deposition. For some medicaments, deposition in the muscle is ideal whereas for other medicaments deposition in the subcutaneous space or intradermal compartment is preferred. Thus, the use of needle-free devices has become of increasing interest, particularly by users such as those requiring mass vaccination, varied depths of delivery, or delivery of variable volumes of medicament.

Conventional low workload needle-free injectors as known in the prior art typically are used once and must be refilled before reuse or must be discarded. Mass administration is very impractical for single use devices because they require a great deal of manipulation between injections. More particularly, prior to each use injection, fresh medicament must be loaded. To load medicament, the user must aseptically transfer fresh medicament from a sterile container to a sterile reservoir within the needle-free device. A concern often associated with aseptic transfer is that to transfer a dose of medicament from aseptic medicament vials, particularly multi-dose vials, requires piercing the vial septum with a sterile needle and syringe, aspirating a dose of medicament into the syringe, then transferring the dose to the needle-free device without introducing contaminating organisms. During this operation, the user must perform additional manipulations to ensure all air is expelled from the injector syringe before administration of medicament. Alternatively, single use needle-free devices employ a unit comprising a pre-sterilized cartridge containing the syringe, nozzle and dose of medicament. To load medicament, the user must change cartridges between use and store fresh cartridges near to the point of use.

Prior art high workload needle-free devices for mass vaccination employ attachments or accessories that are required to provide sufficient energy to drive medicament into or through the skin. Some devices developed for mass vaccination are tethered to bulky components that are cumbersome, or are so awkward for the user that portability is not feasible. A concern often associated with devices that require compressed gas is that a compressed gas cylinder must be either carried in a backpack or firmly anchored to a sturdy structure. A backpack is considered deficient in that it imparts additional weight for the worker leading to premature exhaustion. An added deficiency is a safety hazard posed by valves and pressure regulators that reduce the pressure of compressed gas to a useable range. Valves and pressure regulators protrude from the compressed gas cylinder, and if jarred could result in a ruptured cylinder causing severe injury to workers. By contrast, compressed gas cylinders that are anchored to a sturdy permanent object are not practical in settings in which subjects cannot be readily brought to the device. Thus, a lightweight portable device has become of increasing interest, particularly by those requiring mobility for administration of medicament.

A deficiency of prior art needle-free injection devices is that dose volumes are fixed or limited to a narrow range of adjustment, generally between 0.1 to 1 ml, or are restricted in the total volume that can be delivered in a single administration. To overcome these deficiencies, a user may need to perform multiple injections to deliver the required amount of medicament. A user may also need to employ and maintain a number of devices to assure accessibility to a device that can deliver the desired volume. A user may need to employ a needle and syringe to prepare or transfer medicaments to the device, thereby abrogating the advantages of needle-free injection devices in reducing the relative risk of needle-stick injuries.

Prior art needle-free injectors are often restricted to a preset injection pressure and velocity. More particularly, the depth of injection cannot be adjusted to administer medicaments to different regions in or under the skin. The preferred route of administration for some medicaments is directly into the muscle, or intramuscular. The preferred route of administration for other medicaments is to a region that is not in the muscle, but is below the skin surface, or the subcutaneous space. The preferred route of administration for yet additional medicaments is into the skin, or dermis. Delivery of medicament to a site other than the preferred site can cause improper dosing thereby reducing bioavailability and effectiveness of the medicament.

A concern for prior art needle-free devices is the possibility for accidental discharge or injection when the device is not appropriately pressed against the skin at the injection site, resulting in bruising, improper penetration, or injection site lacerations. For proper administration of medicament, the user selects an injection site on the skin and administers the injection. A concern often associated with administration of medicament using prior art needle-free devices is pressure of the nozzle against the skin and alignment of the orifice to effectively penetrate the skin at the time of injection. Furthermore, prior art needle-free devices can cause skin lacerations if the stream of medicament is prolonged or if the nozzle moves relative to the skin. A deficiency of prior art needle-free injectors is that the configuration does not optimize for pressure at the nozzle-skin interface and can be subject to misalignment.

### SUMMARY OF THE INVENTION

Therefore it is an object of the present invention to provide a needle-fee injection device which overcomes the disadvantages associated with prior art needle-fee injection devices.

The needle-free injection device constructed in accordance with a preferred implementation of the present invention is a portable unit, which automatically and aseptically refills the dose chamber of the syringe between administrations and which will administer additional doses of medicament upon user demand. The needle-free injection device of the present invention is sterilized by passing an acceptable sterilizing agent through the medicament fluid path and is stored with sterilizing solution or sterile physiologic saline in the syringe chamber. Prior to use, sterilizing solution is removed by passing sterile physiologic saline through the syringe. Alternatively, the syringe assembly can be readily uncoupled from the driver assembly, and the syringe portion can be sterilized in a standard autoclave or boiling water. The needle-free injection device of the present device overcomes the deficiencies of prior art needle-free devices because the energy supply for repeated administration is contained within the device making it free from accessories or free from a tether to a compressed gas source. Particularly, the present needle-free device may be used with either hand. The present needle-free device overcomes deficiencies of prior art needle-free devices by providing a triggering mechanism specifically constructed to facilitate preferred contact force and alignment between the nozzle assembly and the skin. Specifically, the syringe housing is linked mechanically to an apparatus that, when released, provides energy to drive medicament at a velocity sufficient to pierce the skin. Additionally, the needle-free injection device of the present invention overcomes deficiencies of fixed-dose prior art needle-free devices as the present needle-free device may be adjusted to deliver varied doses of medicament to varied tissue depths or compartments

Accordingly, a needle-fee injection device for delivery of medicament into or through skin is provided. The needle-fee injection device comprises: a housing having a striker channel and a dose chamber for holding a dose of medicament, the housing further having at least one jet orifice in fluid communication with the dose chamber for delivering the medicament to the skin; a syringe plunger having at least a portion thereof movably disposed in the dose chamber between reload and ejection positions; a striker movable within the striker channel between the reload and ejection positions; energy storage means for storing energy to be delivered to the striker; a driven member under the power of a drive means; at least one engagement member movable between engagement and disengagement positions with the driven member such that engagement of the at least one engagement member with the driven member causes the energy storage means to store energy and disengagement of the at least one engagement member with the driven member allows the stored energy in the energy storage means to be delivered to the striker causing the striker to impact the syringe plunger and eject the medicament from the dose chamber through the at least one jet orifice; and actuation means for selectively engaging and disengaging the at least one engagement member from and to the driven member.

Preferably, the driven member is a jackscrew having a threaded surface, the jackscrew being rotated by the drive means. The drive means is preferably a motor disposed on the housing for driving the driven member.

Preferably, the least one engagement member is a pawl rotatably disposed in the striker between the engagement and disengagement positions with the driven member, the pawl having a threaded surface for mating engagement with the threaded surface of the jackscrew. In which case, the needle-fee injection device further comprises a pawl shuttle slidably disposed in the striker for capturing the pawl and rotating the pawl between the engagement and disengagement positions. Preferably, the needle-fee injection device also further comprises a forward drive housing disposed in the housing and a rear travel limiter disposed in the housing, the pawl shuttle contacting the forward drive housing to slide the pawl shuttle and rotate the pawl into the engaged position, the pawl shuttle contacting the rear travel limiter to slide the pawl shuttle and rotate the pawl into the disengaged position. Preferably, at least one of the forward drive housing and rear travel limiter are adjustably disposed in the housing so as to vary a point at which they contact with the pawl shuttle.

The needle-fee injection preferably further comprises locking means for locking the striker in the ejection position. Preferably, the striker has at least one circumferential ring, wherein the locking means comprises at least one sear rotatably disposed in the housing for lockably engaging with the at least one circumferential ring to lock the striker in the ejection position. In which case, the actuation means preferably comprises automatic release means for automatically releasing the locking means upon application of the housing to the skin with an appropriate force. Preferably, the automatic release means comprises: the housing comprises a syringe housing and a drive housing movable with respect to each other; and at least one actuator linkage rod slidably disposed in the housing, the actuator linkage rod having a first end engaged with a portion of the syringe housing and a second end engaged with the at least one sear, wherein a relative movement of the syringe housing with respect to the drive housing causes the at least one actuator linkage rod to slide and the second end to engage the at least one sear to disengage the at least one sear from the at least one circumferential ring. The needle-fee injection device preferably further comprises biasing means for biasing the drive housing and syringe housing apart.

Preferably, the housing further comprises a nozzle plate having the at least one jet orifice, the nozzle plate having two or more facets, wherein the at least one jet orifice comprises a jet orifice corresponding to each of the two or more facets. The nozzle plate preferably further has a plenum chamber in communication with both the dose chamber and each of the jet orifices.

The needle-fee injection device preferably further comprises an outflow check valve disposed in a fluid path between the dose chamber and the at least one jet orifice for restricting a flow of fluid back into the dose chamber from the at least one jet orifice.

Preferably, the syringe plunger has a stepped portion corresponding to a stepped portion of the housing, interference of the stepped portions limiting the extent of movement of the portion of the syringe plunger into the dose chamber. In which case, the needle-fee injection device further comprises biasing means disposed between the stepped portions for biasing the syringe plunger into the ejection position.

The needle-fee injection device preferably further comprises an intake check valve having a fluid path in fluid communication with a source of medicament and the dose chamber, the intake check valve restricting a flow of medicament from the dose chamber back to the source of medicament.

Preferably, the energy storage means is a compression spring disposed in the striker channel between the striker and a portion of the housing.

The needle-fee injection device preferably further comprises a fluid seal disposed between a surface of the dose chamber and a corresponding surface of the syringe plunger for preventing a fluid flow between the surface of the dose chamber and the surface of the syringe plunger.

Also provided is a method for delivering medicament into or through skin with a reusable needle-fee injection device, the method comprising: (a) drawing a medicament into a dose chamber of the device by withdrawing a syringe plunger at least partially disposed in the dose chamber from a reload position to an ejection position; (b) moving a striker between the reload and ejection positions; (c) storing energy in an energy storage means when the striker is moved to the ejection position; (d) ejecting medicament from the dose chamber through at least one jet orifice upon pressing a distal portion containing the at least one jet orifice against the skin; and (e) repeating steps (a) through (e) with the reusable needle-free injection device.

Still provided is a method for delivering medicament into or through skin with a needle-fee injection device. The method comprises: drawing a medicament into a dose chamber of the device by withdrawing a syringe plunger at least partially disposed in the dose chamber from a reload position to an ejection position; moving a striker between the reload and ejection positions; storing energy in an energy storage means when the striker is moved to the ejection position; driving a driven member; selectively engaging and disengaging at least one engagement member from the driven member such that engagement of the at least one engagement member with the driven member causes the energy storage means to store energy and disengagement of the at least one engagement member with the driven member allows the stored energy in the energy storage means to be delivered to the striker causing the striker to impact the syringe plunger; and ejecting the medicament from the dose chamber through at least one jet orifice.

Preferably, the drawing comprises biasing the syringe plunger proximally to withdraw the syringe plunger to the ejection position.

The driving preferably comprises rotating the driven member. In which case, the selectively engaging and disengaging at least one engagement member from the driven member comprises: engaging the at least one engagement member with the driven member; moving the striker against the energy storage means to the ejection position to store energy in the energy storage means; engaging a locking means with at least a portion of the striker; and releasing engagement of the at least one engagement member from the driven member. Preferably, the ejecting also comprises: depressing a trigger to disengage an interlock; and applying a distal portion of the needle-free injection device containing the at least one jet orifice to a desired injection site with a predetermined amount of pressure.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 is a side view depicting the mechanisms and component assemblies of a preferred implementation of a needle-fee injection device.

FIG. 2 is a top view depicting the mechanisms and individual components of the needle-fee injection device of FIG. 1.

FIG. 3 is a detailed view depicting components of the nozzle assembly, outflow check valve assembly, and nozzle coupler of the needle-fee injection device of FIG. 1.

FIG. 3a is a detailed view depicting components of the intake check valve assembly of the needle-fee injection device of FIG. 1.

FIG. 4 is a top view depiction of the needle-fee injection device of FIG. 2 showing mechanism(s) provided therein at the end of the ejection phase of the operation cycle, wherein the dose chamber volume is reduced by the selected dose, residual mainspring energy holds the striker at its forward limit of travel, and the pawl is forced to engage the jackscrew by contact of the pawl shuttle against the forward drive housing.

FIG. 5 is a top view depiction of the needle-fee injection device of FIG. 2 showing mechanism(s) provided therein during the reloading phase of the operation cycle, wherein the pawl is engaged with the jackscrew and held in that position by contact of the pawl shuttle with the shuttle spring detent, rotation of the jackscrew against the pawl causes retraction of the striker against the mainspring, sears are in contact with the striker, and are free to engage circumferential rings as the striker is displaced rearward, and the plunger is being moved rearward by force of the plunger return spring, thus, filling the dose chamber.

FIG. 6 is a top view depiction of the needle-fee injection device of FIG. 2 showing mechanism(s) provided therein when ready for use, wherein the striker is held retracted against the mainspring by two sears, the interlock prevents disengagement of the sear engaged from the striker, the pawl is disengaged from the jackscrew by contact of the pawl shuttle against the rear travel limiter, the jackscrew is free to rotate, the plunger is at the rearward limit of its travel, and the dose chamber is filled.

FIG. 7 is a top view depiction of the needle-fee injection device of FIG. 2 showing mechanism(s) provided therein during the ejection phase of the operation cycle, wherein the interlock is disengaged from the sears by the trigger lever, the syringe housing assembly is displaced rearward against the standoff tensioning spring, displacing the trigger linkage rods rearward against the sears, the sears are disengaged from the striker, releasing the striker to accelerate forward against the plunger, the pawl is disengaged from the jackscrew and held in that position by contact of the pawl shuttle with the shuttle spring detent, the jackscrew is free to rotate, and the selected dose volume is expelled from the dose chamber through the outflow check valve and nozzle assemblies.

### DETAILED DESCRIPTION OF THE PREFERRED EMBODIMENT

Although this invention is applicable to numerous and various types of patients, it has been found particularly useful in the environment of veterinary subjects. Therefore, without limiting the applicability of the invention to veterinary subjects, the invention will be described in such environment. Those skilled in the art will appreciate that the needle-free injection device of the present invention may also be useful on human subjects, particularly where there is no need for sterilization of the device between uses. However, those skilled in the art will also appreciate that the needle-free injection device of the present invention can be easily modified for such sterilization according to cleaning and sterilization principles known in the art.

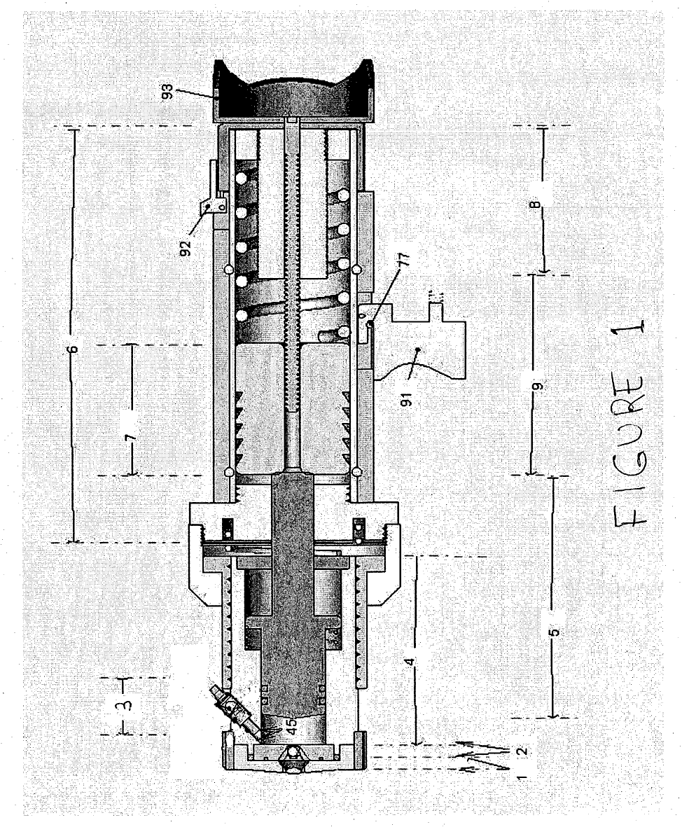

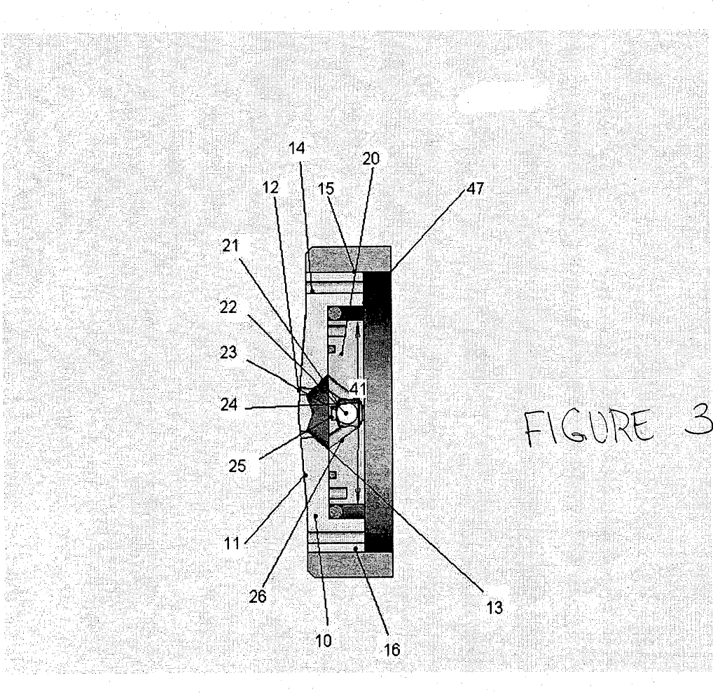

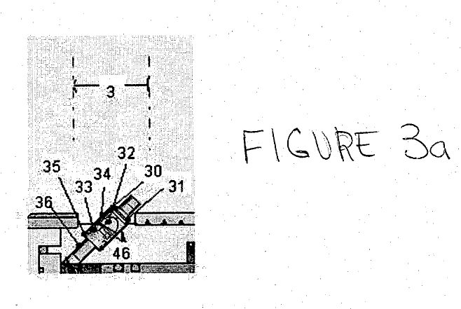

Referring now to FIGS. 1-3, a nozzle assembly 1 contains multiple jet orifices 12 to facilitate very rapid injection of liquid medicament from a dose metering chamber 42. As described, the nozzle assembly 1 can be reversed in its coupling to allow back-flushing for cleaning of orifices. Multiple slightly divergent jets enhance subcutaneous and intradermal delivery of medicament, and reduce the time of delivery proportional to the number of orifices employed. Referring now specifically to FIG. 3, the nozzle assembly 1 comprises a nozzle disk 10 which is preferably a single piece of stainless steel or approved medical grade material and which contains 2 to 6 uniformly sized facets 11 milled in a radial array at a shallow angle of approximately 5 to 15 degrees. Each orifice 12 is preferably in the range of 0.1 to 0.5 millimeters in diameter, and is drilled perpendicular to its respective facet such that the orifices converge in a plenum chamber 13 on the reverse aspect of the nozzle disk 10. The facets 11 and multiple orifices 12 help to minimize relative movement of the nozzle assembly 1 relative to the skin and to deliver larger amounts of medicament in a short time, both of which tend to decrease the possibility of lacerating the skin at the site of the injection. The nozzle disk 10 further contains two holes 14 approximately two millimeters in diameter near the periphery at opposite ends of a line bisecting the nozzle disk 10, which serve as the receptacle for a 2-pin spanner wrench (not shown). The circumference of the nozzle disk 10 constitutes a threaded surface 15 for mating to a nozzle coupler 16.

Referring back to FIGS. 1-3, a stainless steel outflow check valve assembly 2 is affixed between the dose metering chamber 42 and the nozzle plenum chamber 13 to prevent aspiration through the nozzle orifices 12 during a filling cycle. Referring again specifically to FIG. 3, the check valve assembly 2 consists of a disk-shaped housing 20 containing a tapered seat 21, a ball 22 of sufficient diameter to occlude flow when in contact with the seat 21, a spring 23 of sufficient strength to hold the ball 22 in contact with the seat 21 against fluid pressure of up to approximately 10 psi, and a retainer 24 which contains a recess 25 for the spring 23 and multiple flow channels 26. The outflow check valve assembly 2 is preferably capable of withstanding instantaneous pressures exceeding 400 bar (6,000 psi), and has a nominal cracking (opening) pressure of approximately 10 psi. As will be apparent from the below description, the outflow check-valve assembly 2 prevents drawing of fluids, such as air or medicament, back into the dose chamber 42 as the syringe plunger 50 is withdrawn.

Referring now to FIGS. 1 and 3a, a stainless steel intake check valve assembly 3 is threaded into the syringe housing 40 to prevent backflow and loss of pressure during an ejection cycle. The check valve assembly 3 consists of a housing 30 containing a tapered seat 31, a ball or truncated cone 32 of sufficient diameter to occlude all flow when in contact with the seat 31, a spring 33 of sufficient strength to hold the ball 32 in contact with the seat 31 against fluid pressure of up to approximately 2 psi, and a retainer 34 which contains a recess 35 for the spring 33 and multiple flow channels 36. The intake check valve assembly 3 is preferably capable of preventing backflow at instantaneous pressures exceeding 400 bar (6,000 psi), and has a nominal cracking (opening) pressure of approximately 2 psi.

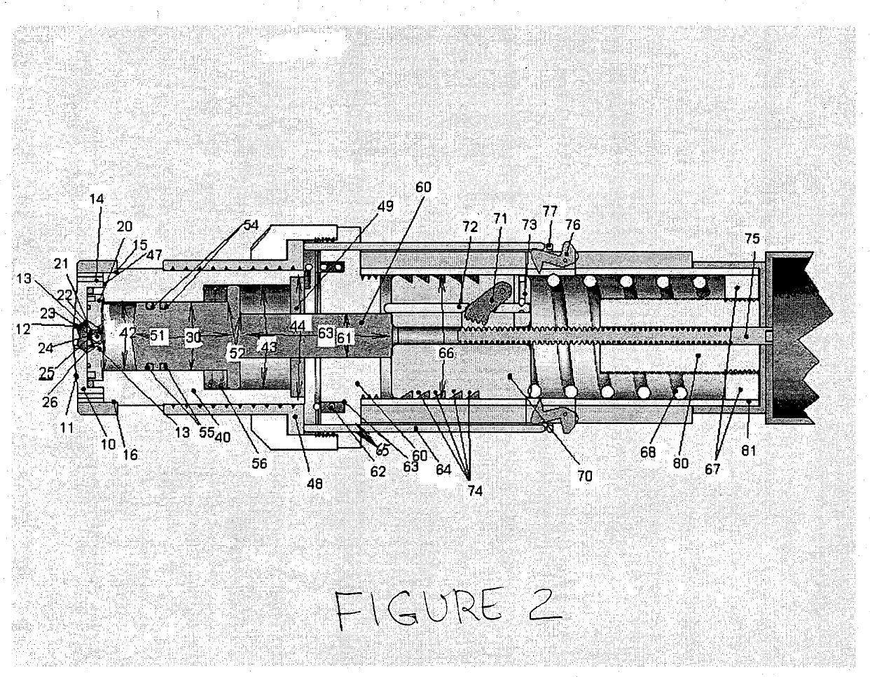

Referring back to FIGS. 1 and 2, a syringe assembly 4 comprises a syringe housing 40, which is preferably fabricated from a single cylindrical piece of stainless steel or approved medical grade material that has the following features. The housing 40 contains a stepped cylindrical cavity about its longitudinal centerline, such that an outflow check valve bore 41 at the most forward portion is of slightly larger diameter than the dose metering chamber bore 42, which in turn is of smaller diameter than the plunger spring bore 43 at the rearward portion. The most rearward portion of the bore constitutes an internally threaded retainer bore 44 of slightly larger internal diameter than the plunger spring bore 43. The forward portion of the syringe housing 40 contains a dose feed channel bore 45 drilled perpendicular to the long axis and at an angle to the cross-sectional axis, such that the feed channel bore 45 angles forward to intersect the dose metering chamber bore 42 at its extreme forward portion. The outer (peripheral) portion of the dose feed channel bore 45 is counter-bored to larger diameter to constitute the intake check valve receptacle bore 46 and is threaded internally to accept the high pressure intake check valve assembly 3. The outer diameter of the forward end of the syringe housing 4 is preferably machined to form a recess 47 to mate with the nozzle assembly 1 and coupler 16. The rearward peripheral portion of the housing 4 is externally threaded 48 to accommodate a dose adjustment sleeve 49 with a flange for mating to a forward drive housing 6 by way of a locking collar. The length of the dose chamber bore 42 is sufficient to provide the desired volume within the dose chamber 42, in addition to accommodating the forward portion and O-rings 55 of the plunger assembly 5. The length of the plunger spring bore 43 is sufficient to accommodate a plunger return spring 56, the plunger shoulder 52, and a plunger retainer ring 57.

Referring now to FIG. 2, the syringe plunger assembly 5 is preferably a single piece of stainless steel or approved medical grade material that has the following features. A plunger 50 is a complex stepped cylindrical part with three separate diameters, as follows. A dose chamber portion 51 of the plunger 50 is of a diameter suitable to slide within the dose chamber bore 42. A mid portion of the plunger 50 constitutes a shoulder 52 of diameter suitable to slide within the plunger spring bore 43, and to accommodate the plunger return spring 56 that surrounds part of the dose chamber portion 51 of the plunger 50. A rearward retainer portion 53 of the plunger 50 is of smaller diameter than the shoulder 52 and dose chamber 51 portions, and is of a diameter suitable to slide within the plunger retainer ring 57. The plunger 50 contains two or more circumferential O-ring channels 54 preferably machined approximately 1 millimeter from its forward end, and approximately 1 millimeter apart. The O-ring channels 54 accept suitable O-rings 55 to provide a required pressure seal within the dose chamber 42 under discharge.

The length of the dose chamber portion 51 of the plunger 50 is sufficient to encompass the length of the dose chamber 42 and the length of the compressed plunger return spring 56. The length of the shoulder portion 52 is adequate to provide material strength as a seat for the plunger return spring 56 and to provide a sliding surface against the plunger spring bore 43 of the syringe housing 40. The length of the retainer portion 53 is equal to the thickness of the plunger retainer ring 57 plus the length of a forward drive housing 60 so that none of the retainer portion 53 of the plunger 50 protrudes rearward of the forward drive housing 60 when the plunger 50 is at the limit of its forward excursion. The plunger return spring 56 provides adequate rearward force on the plunger assembly 5 to draw liquid into the dose chamber through the intake check valve assembly 3.

The assembled syringe 5 is mated to a drive assembly 6 by way of a locking collar that engages a threaded surface at the forward end of the forward drive housing 60. The drive assembly 6 could use any available power source capable of accelerating an inertial element (striker assembly) 7 to transfer the required amount of energy to the syringe plunger assembly 5. Power sources may be mechanical spring, compressed gas spring, combustion of fuel, pyrotechnic discharge, electromagnetic field (electrical solenoid), etc.

The syringe assembly 5 is held by spring tension at a nominal gap (standoff) of approximately 2 to 5 millimeters from the forward face of the drive housing 60. The retainer portion of the plunger 53 extends rearward through a plunger bore 61 in the forward drive housing 60. A standoff tensioning spring 62 is retained in a spring channel 63, and provides adequate standoff force to assure optimal contact pressure between the nozzle 12 and skin surface during the ejection cycle. When the device is pressed against the desired injection site on an animal with the manual trigger lever 91 depressed, the syringe plunger assembly 5 compresses the standoff tensioning spring 62, allowing actuator linkage rods 64 to be displaced rearward through linkage channels 65 against sears 76 that restrain an inertial element (striker assembly) 7. This constitutes a safety device that prevents discharge of the injector unless the device is properly placed against the animal, and allows discharge as soon as the device is appropriately positioned. A safety interlock 77 is controlled by a manually operated lever (trigger) 91 in the handle 90. The safety interlock 71 prevents the linkage rod 64 from displacing the sear 76 unless the manual trigger lever 91 is depressed. A spring holds the trigger lever 91 and interlock 71 in the engaged position by default. When the interlock 71 is disengaged, the sear 76 can be released by pressure of the actuator linkage rod 64 when the device is pressed against the desired injection site with sufficient force to compress the standoff tensioning spring 62. Upon release of the sear(s) 76, the striker assembly 7 is projected forward within the striker channel 66 to impact the syringe plunger assembly 5 with sufficient force to generate a peak pressure within the fluid-filled dose chamber of approximately 345 bar (5,000 psi)

Volume of the dose of medicament delivered is controlled by position of the dose adjustment sleeve 49 on the syringe housing 40. The thread pitch of the threaded interface 48 is preferably set so that one complete rotation of the dose adjustment sleeve 49 moves the syringe housing 40 relative to the forward drive housing 60 by a distance calculated to equal 0.1 mL volume in the dose metering chamber. At the nominal “zero volume” setting, the syringe plunger assembly 5 would be shifted forward to the point at which, with the dose chamber 42 filled, the rearward face of the syringe plunger assembly 5 is flush with the rearward face of the forward drive housing 60 when the syringe plunger assembly 5 is maximally compressed against the standoff tensioning spring 62. At that nominal “zero volume” setting, the striker assembly 7 would not displace the plunger assembly 5, and the total volume of medicament would be retained within the dose chamber 42 at completion of the ejection cycle. Conversely, at the nominal “maximal volume” setting, the syringe plunger assembly 5 would be shifted rearward relative to the drive housing 60 to the point at which the rearward face of the syringe plunger assembly 5 extends some maximal distance behind the rearward face of the forward drive housing 60 when the syringe plunger assembly 5 is maximally compressed against the standoff tensioning spring 62. That maximal distance would correspond to the plunger excursion required to expel the entire usable volume of medicament within the dose chamber 42. Intermediate settings of the dose adjustment sleeve 49 would limit the distance of plunger excursion to deliver some set portion of the usable volume of medicament within the dose chamber 42.

One illustrated configuration of the drive assembly 6 uses a mechanical (coil) main spring 68 of progressive compression strength to accelerate the striker assembly 7 against the syringe plunger assembly 5. The amount of energy transferred to the syringe plunger assembly 5 is adjustable by controlling the degree of main spring compression and the distance (gap) across which the striker 70 is accelerated before contacting the plunger assembly 5. The main spring 68 is compressed against the main spring seat 67 at the rear of the striker channel 66.

The striker assembly 7 is retracted by a rotating threaded shaft (jackscrew) 75 that extends through the center of the striker 70. The jackscrew is engaged by a pawl 71 within the striker 70, causing the striker assembly 7 to travel rearward against pressure of the main spring 68. When the striker 70 is retracted rearward to the desired position, the pawl shuttle 72 comes into contact with a rear travel limiter 80, causing the shuttle 72 to move within the striker 72, disengaging the pawl from the rotating jackscrew 75. A spring detent 73 locks the shuttle 72 in either forward or rearward position, holding the pawl 71 in “disengaged” or “engaged” positions, respectively. Simultaneously, one of the circumferential rings 74 on the striker 70 is engaged by two sears 76 that inhibit its forward motion, effectively holding the striker 70 rearward against the desired main spring 68 tension (cocked) with the pawl 71 disengaged from the freely rotating jackscrew 75. When the sear 76 is disengaged from the striker assembly 7 (triggered), the energy of the compressed main spring 68 is released, accelerating the striker 70 forward, contacting and displacing forward the plunger assembly 5. Forward travel of the striker 70 is limited by the forward drive housing 60 at the front of the striker channel 66, so that the striker 70 can not impart energy to the plunger 50 forward of the point of maximal desired plunger excursion. As the striker 70 contacts the forward stop, the shuttle 72 is displaced rearward within the striker 70, forcing the pawl 71 to engage the rotating jackscrew 75. The jackscrew 75 is turned by a motor 93 that is attached to a motor mount 82 at the rear portion of the rear travel limiter assembly 8. The motor 92 is preferably an electric motor powered by a battery pack 94 in the handle/housing assembly 9, but may be other types of motors known in the art, such as a pneumatic motor powered by a supply of compressed gas. Alternatively, the electric motor may be powered by an electrical cord connected to a power supply. By virtue of notches machined into the striker 70 and spring seat faces, the main spring 68 prevents rotation of the striker 70 within the striker channel 66 while the striker assembly 7 is being retracted by force of the rotating jackscrew 75 against the pawl 71.

Adjustment of energy transferred to the syringe plunger assembly 5 is accomplished by controlling the degree of main spring 68 compression and the distance (gap) across which the striker 70 is accelerated before contacting the syringe plunger assembly 5. The rear travel limiter 80 can be moved longitudinally within the striker channel 66 by rotating it along its threaded interface with the main spring seat 67. The thread pitch of that interface is preferably set so that one complete rotation of the rear travel limiter 80 moves its forward face a distance equal to the distance between circumferential rings 74 on the striker 70. In that way, one rotation counterclockwise retracts the face of the rear travel limiter 80 the precise distance required to allow the sear 76 to engage the next ring 74 on the striker 70, and therefore to restrain the striker assembly 7 at one step greater distance from the plunger 50 with the main spring 68 compressed additionally by that same distance. The accompanying diagrams depict a series of five such rings 74 on the striker 70, allowing for adjustment of the cocked striker-to-plunger gap from zero (or minimal) to some maximal distance, and adjustment of cocked main spring 68 tension from nominal minimal to nominal maximal pressure. A locking lever 92 on the handle/housing assembly 9 engages a slot in the external sleeve 81 of the rear travel limiter assembly 8 to ensure proper rotational alignment during operation.

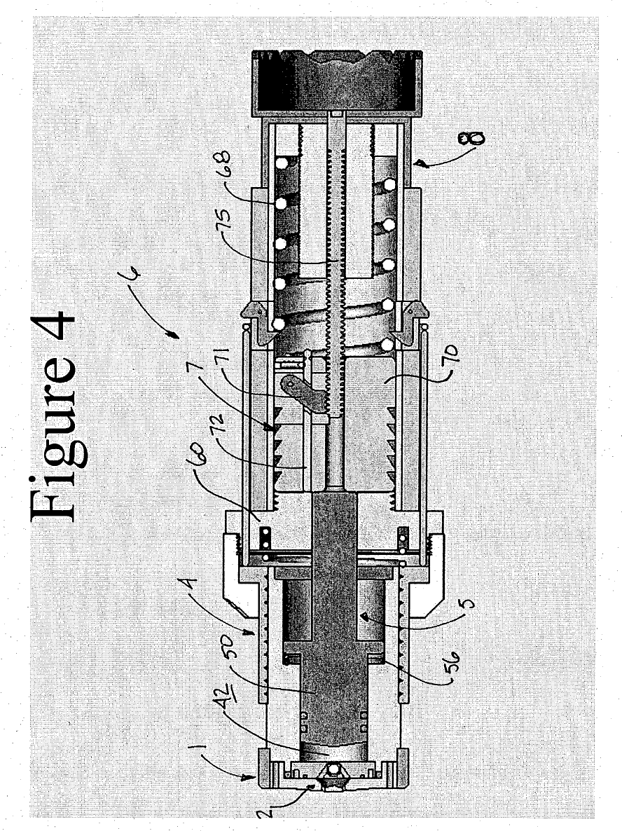

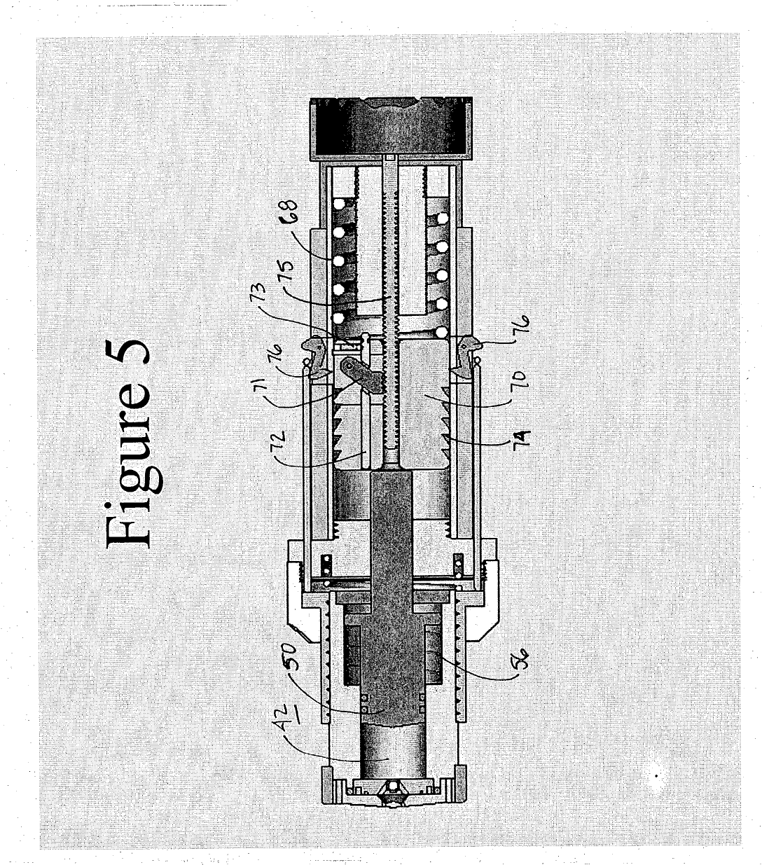

The operation of the needle-fee injection device of the present invention will now be described with reference to FIGS. 4-7. FIG. 4 is a top view depiction of the needle-fee injection device of FIG. 2 showing the mechanism(s) provided therein at the end of the ejection phase of the operation cycle. At the end of the ejection phase, the dose chamber 42 volume is reduced by the selected dose, residual main spring 68 energy holds the striker 70 at its forward limit of travel, and the pawl 71 is forced to engage the jackscrew 75 by contact of the pawl shuttle 72 against the forward drive housing 60. In FIG. 5 the needle-fee injection device is shown during the reloading phase of the operation cycle. During the reloading phase, the pawl 71 is engaged with the jackscrew 75 and held in that position by contact of the pawl shuttle 72 with the shuttle spring detent 73. Rotation of the jackscrew 75 against the pawl 71 causes retraction of the striker 70 against the mainspring 68. Further, the sears 76 are in contact with the striker 70 and are free to engage the circumferential rings 74 as the striker 70 is displaced rearward. The plunger 50 is also moved rearward by force of the plunger return spring 56, thus, filling the dose chamber 42.

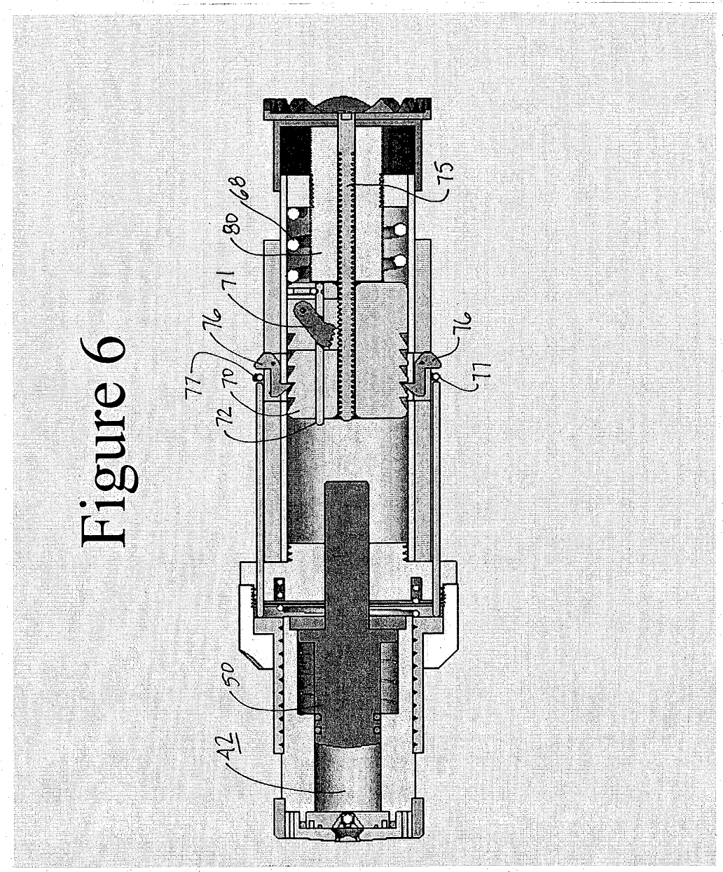

In FIG. 6 the needle-fee injection device is shown ready for use. The striker 70 is held retracted against the main spring 68 by the two sears 76, the interlock 77 prevents disengagement of the sears 76 engaged with the striker 70, the pawl 71 is disengaged from the jackscrew 75 by contact of the pawl shuttle 72 against the rear travel limiter 80. Further, the jackscrew 75 is free to rotate, the plunger 50 is at the rearward limit of its travel, and as a result, the dose chamber 42 is filled through the intake check valve assembly 3.

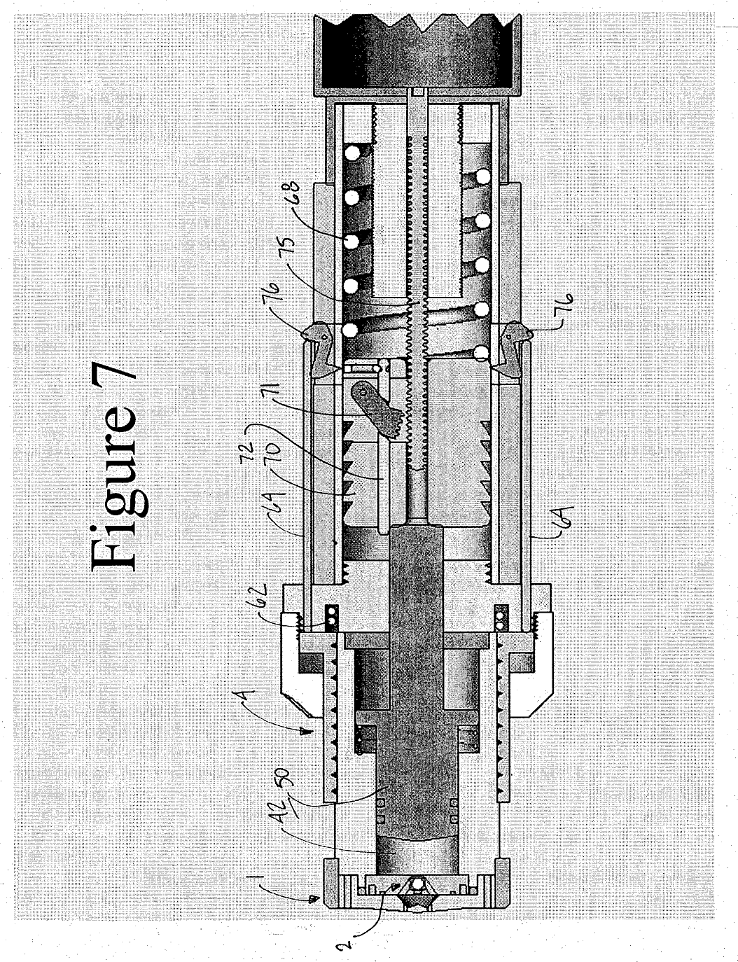

In FIGS. 1 and 7 the needle-fee injection device is shown during the ejection phase of the operation cycle. In the ejection phase, the interlock 77 is disengaged from the sears 76 by the trigger lever 91. When the nozzle assembly 1 is pressed against the skin, the syringe housing assembly 4 is displaced rearward against the standoff tensioning spring 62, displacing the trigger linkage rods 64 rearward against the sears 76. The sears 76 are disengaged from the striker 70, releasing the striker 70 to accelerate forward against the plunger 50. The pawl 71 is disengaged from the jackscrew 75 and held in the disengaged position by contact of the pawl shuttle 72 with the shuttle spring detent 73 and the jackscrew 75 is free to rotate. The selected dose volume is expelled from the dose chamber 42 through the outflow check valve 2 and nozzle assemblies 1. At the end of the ejection phase (shown in FIG. 4), the needle-fee injection device enters the reloading phase (shown in FIG. 5) and the process can repeat until the supply of medicament is exhausted or until injections are no longer needed. Although the operation of the needle-free injection device is preferred and described as requiring both a pressure of the nozzle assembly 1 against the skin and pressure applied to the trigger lever 91, those skilled in the art will appreciate that either mechanism can be initiated first to operate the device. For example, the needle-free injection device can be automatically triggered to eject the medicament solely by the application of a sufficient pressure of the nozzle assembly 1 against the skin if the trigger lever 91 is already being held back.

Those skilled in the art will appreciate that the intake check valve assembly 3 is preferably positioned between the dose chamber 42 and a fluid medicament reservoir to assure that the total energy of injection is delivered to the jet orifice 12 and that the entire desired dose is expelled through the jet orifice 12. The outflow check valve assembly 2 is preferably positioned between the dose chamber 42 and the jet orifice 12 to assure that air does not flow into the dose chamber 42 while the plunger 50 is drawn back to aspirate fluid into the dose chamber 42. The jet orifice 12 creates a stream of fluid medicament, which when traveling at high velocity, pierces the skin permitting delivery of medicament into or through the skin.

As described above with regard to FIGS. 4-7, the plunger 50 repositions forward and rearward in the syringe housing 40. Rearward movement of the plunger 50 is driven by the spring 56. The tension of the spring 56 is sufficient to overcome resistance that is created by flow of the medicament through the intake check valve assembly 3 and the friction created by the o-ring seals 55. O-ring seals 55 are required to assure fluid flows through the jet orifice 12 with the forward movement of the plunger 50, and into the dose chamber 42 with the rearward movement of the plunger 50. Forward movement of the plunger 50 occurs during injection and is driven by the striker assembly 7. In the preferred embodiment of the present invention, the entire syringe assembly 5 can be removed for cleaning or maintenance. The syringe assembly 5 can be further replaced with another syringe assembly 5 constructed to deliver a dose volume that may be greater than or may be smaller than the dose volume of the syringe assembly 5 that was removed.

In the operation of the present needle-free mass injection device, an amount of force is required of the user to result in a preferred contact between the nozzle jet orifice 12 and the skin. The force applied by the user must overcome resistance created by a standoff tensioning spring 62 that situates the syringe at the outward most position from the device. Application of force to the syringe housing assembly 4, by positioning the nozzle 12 against the skin and pushing the device toward the skin, effects release of the striker 70 within the drive housing 60 via the actuator linkage rod 64. The force applied by the user to the syringe housing assembly 4 drives the actuator linkage rod 64 to release the sear 76 that restrains the striker 70. Once released, the striker 70 is propelled forward by the main spring 68. The striker 70 impacts the plunger 50 driving the fluid through the jet orifice 12, piercing the skin, followed by deposition of medicament in and or through the skin.

In the preferred implementation of the present invention, the forward most travel of the striker 70 engages the pawl 71, which is affixed to the striker 70. The pawl 71 engages the jackscrew 75 by direct contact of the pawl shuttle 72 against the forward drive housing 60 as forward movement of the striker 70 is brought to a halt. The pawl shuttle 72, when forced rearward by contact with the drive housing 60, pushes the pawl 71 from the disengaged position to the engaged position. Once the pawl 71 is engaged, the rotating jackscrew 75 retracts the striker 70 rearward against the tension of the main spring 68. Rearward movement of the striker 70 ends when the pawl shuttle 72 contacts a rear travel limiter 80. The travel limiter 80 can be adjusted to disengage the pawl 71 at varied distances of travel.

This, in effect, facilitates varied amounts of stored energy. The amount of stored energy determines the velocity of injection. By adjusting the amount of stored energy, therefore, a user can adjust the velocity of injection to accommodate differences in skin thickness or depth of injection. In particular, an intramuscular injection requires more injection energy than injection into the subcutaneous space. In the absence of force against the nozzle assembly 1, the two sears 76 engage the striker 70, restraining the striker 70 in the preferred rearward position. Simultaneously, the pawl shuttle 72 contacts the rear travel limiter 80, which then pushes the pawl 71 from the engaged position to the disengaged position. The pawl 71 is held in the disengaged position by a shuttle spring detent 73, which in turn, prevents contact between the jackscrew 75 and the pawl 71 during operation until the pawl shuttle 72 impacts against the forward drive housing 60.

In the preferred implementation of the present needle-free injector, the jackscrew 75 attaches to a battery-powered electric motor 93. Activation of the electric motor 93 causes the jackscrew 75 to rotate. A switch mounted on the housing causes the motor 93 to rotate when the switch is in the on position and stops the motor 93 from rotating when the switch is in the off position.

Further in accordance with the present invention, there is provided a method for mass injection using a needle-free device having the above-described structural features. Medicament is preferably introduced into the previously sterilized syringe by making an aseptic connection between the intake check valve assembly 3 and medicament container. The pressure, applied by the user, of the nozzle assembly 1 against the skin serves as the trigger to perform an injection. The striker assembly 7, main spring 68, pawl 71, and sears 76 serve to facilitate multiple injections of medicament. The striker assembly 7, main spring 68, pawl 71, and sears 76 further serve to facilitate depth of injection. The plunger 50 and jet orifice 12 serve to drive medicament into and or through the skin.

Due to the inventive arrangement of the novel needle-free injection device of the present invention the advantages offered by such resides at least in:

(a) A mechanical means of compressing a coil spring that allows unencumbered release of the stored energy to accelerate the inertial element (striker) against the syringe plunger. Rotation of the pawl away from the jackscrew by action of the pawl shuttle removes mechanical interference from forward flight of the shuttle, allowing optimal recovery of the stored energy;

(b) Contact of the pawl shuttle with the forward travel limiter (drive housing) at the end of the ejection cycle automatically causes the pawl to engage the jackscrew, causing movement of the striker against the spring, storing energy for the next operation cycle;

(c) The energy available to accelerate the inertial element (striker) is adjustable for optimal delivery of a range of dose volume and/or a range of skin thickness. The energy adjustment is accomplished both by altering the length of compression of a progressive coil spring and by altering the distance of free acceleration (gap) between the striker and the syringe plunger;

(d) Optimal contact force and alignment of the nozzle with the skin is assured by the means of actuation (triggering). Specifically, the device cannot be discharged unless it is pressed against the desired injection site with sufficient force to compress the standoff spring (biasing means) and slide the actuator linkage rod(s) to rotate the sear(s) (locking means) to disengage the striker. The interlock feature enables actuation, but does not discharge the device in the absence of the required contact pressure; and

(e) The multiple orifice nozzle design is unique in that it facilitates very rapid ejection of the desired dose and delivers a larger distribution pattern of medicament in the injection site tissue. The faceted design facilitates machining (manufacture) of divergent jet orifices and provides a means of delivering medicament in a larger volume of deposition thus, the stream of medicament is delivered in a shorter time to reduce the possibility of skin lacerations.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A needle-fee injection device for delivery of medicament into or through skin, the needle-fee injection device comprising:
a housing having a striker channel and a dose chamber for holding a dose of medicament, the housing further having at least one jet orifice in fluid communication with the dose chamber for delivering the medicament to the skin;
a syringe plunger having at least a portion thereof movably disposed in the dose chamber between reload and ejection positions;
a striker movable within the striker channel between the reload and ejection positions;
energy storage means for storing energy to be delivered to the striker;
a driven member under the power of a drive means;
at least one engagement member movable between engagement and disengagement positions with the driven member such that engagement of the at least one engagement member with the driven member causes the energy storage means to store energy and disengagement of the at least one engagement member with the driven member allows the stored energy in the energy storage means to be delivered to the striker causing the striker to impact the syringe plunger and eject the medicament from the dose chamber through the at least one jet orifice; and
actuation means for selectively engaging and disengaging the at least one engagement member from and to the driven member.

2. The needle-fee injection device of claim 1, wherein the driven member is a jackscrew having a threaded surface, the jackscrew being rotated by the drive means.

3. The needle-fee injection device of claim 2, wherein the drive means is a motor disposed on the housing for driving the driven member.

4. The needle-fee injection device of claim 2, wherein the least one engagement member is a pawl rotatably disposed in the striker between the engagement and disengagement positions with the driven member, the pawl having a threaded surface for mating engagement with the threaded surface of the jackscrew.

5. The needle-fee injection device of claim 4, further comprising a pawl shuttle slidably disposed in the striker for capturing the pawl and rotating the pawl between the engagement and disengagement positions.

6. The needle-fee injection device of claim 5, further comprising a forward drive housing disposed in the housing and a rear travel limiter disposed in the housing, the pawl shuttle contacting the forward drive housing to slide the pawl shuttle and rotate the pawl into the engaged position, the pawl shuttle contacting the rear travel limiter to slide the pawl shuttle and rotate the pawl into the disengaged position.

7. The needle-fee injection device of claim 6, wherein at least one of the forward drive housing and rear travel limiter are adjustably disposed in the housing so as to vary a point at which they contact with the pawl shuttle.

8. The needle-fee injection device of claim 1, further comprising locking means for locking the striker in the ejection position.

9. The needle-fee injection device of claim 8, wherein the striker has at least one circumferential ring, wherein the locking means comprises at least one sear rotatably disposed in the housing for lockably engaging with the at least one circumferential ring to lock the striker in the ejection position.

10. The needle-fee injection device of claim 9, wherein the actuation means comprises automatic release means for automatically releasing the locking means upon application of the housing to the skin with an appropriate force.

11. The needle-fee injection device of claim 10, wherein the automatic release means comprises:
the housing comprises a syringe housing and a drive housing movable with respect to each other; and
at least one actuator linkage rod slidably disposed in the housing, the actuator linkage rod having a first end engaged with a portion of the syringe housing and a second end engaged with the at least one sear, wherein a relative movement of the syringe housing with respect to the drive housing causes the at least one actuator linkage rod to slide and the second end to engage the at least one sear to disengage the at least one sear from the at least one circumferential ring.

12. The needle-fee injection device of claim 11, further comprising biasing means for biasing the drive housing and syringe housing apart.

13. The needle-fee injection device of claim 1, wherein the housing further comprises a nozzle plate having the at least one jet orifice, the nozzle plate having two or more facets, wherein the at least one jet orifice comprises a jet orifice corresponding to each of the two or more facets.

14. The needle-fee injection device of claim 13, wherein the nozzle plate further has a plenum chamber in communication with both the dose chamber and each of the jet orifices.

15. The needle-fee injection device of claim 1, further comprising an outflow check valve disposed in a fluid path between the dose chamber and the at least one jet orifice for restricting a flow of fluid back into the dose chamber from the at least one jet orifice.

16. The needle-fee injection device of claim 1, wherein the syringe plunger has a stepped portion corresponding to a stepped portion of the housing, interference of the stepped portions limiting the extent of movement of the portion of the syringe plunger into the dose chamber.

17. The needle-fee injection device of claim 16, further comprising biasing means disposed between the stepped portions for biasing the syringe plunger into the ejection position.

18. The needle-fee injection device of claim 1, further comprising an intake check valve having a fluid path in fluid communication with a source of medicament and the dose chamber, the intake check valve restricting a flow of medicament from the dose chamber back to the source of medicament.

19. The needle-fee injection device of claim 1, wherein the energy storage means is a compression spring disposed in the striker channel between the striker and a portion of the housing.

20. The needle-fee injection device of claim 1, further comprising a fluid seal disposed between a surface of the dose chamber and a corresponding surface of the syringe plunger for preventing a fluid flow between the surface of the dose chamber and the surface of the syringe plunger.

21. A method for delivering medicament into or through skin with a reusable needle-fee injection device, the method comprising:
(a) drawing a medicament into a dose chamber of the device by withdrawing a syringe plunger at least partially disposed in the dose chamber from a reload position to an ejection position;
(b) moving a striker between the reload and ejection positions;
(c) storing energy in an energy storage means when the striker is moved to the ejection position;
(d) ejecting medicament from the dose chamber through at least one jet orifice upon pressing a distal portion containing the at least one jet orifice against the skin; and
(e) repeating steps (a) through (e) with the reusable needle-free injection device.

22. A method for delivering medicament into or through skin with a needle-fee injection device, the method comprising:
drawing a medicament into a dose chamber of the device by withdrawing a syringe plunger at least partially disposed in the dose chamber from a reload position to an ejection position;
moving a striker between the reload and ejection positions;
storing energy in an energy storage means when the striker is moved to the ejection position;
driving a driven member;
selectively engaging and disengaging at least one engagement member from the driven member such that engagement of the at least one engagement member with the driven member causes the energy storage means to store energy and disengagement of the at least one engagement member with the driven member allows the stored energy in the energy storage means to be delivered to the striker causing the striker to impact the syringe plunger; and
ejecting the medicament from the dose chamber through at least one jet orifice.

23. The method of claim 22, wherein the drawing comprises biasing the syringe plunger proximally to withdraw the syringe plunger to the ejection position.

24. The method of claim 22, wherein the driving comprises rotating the driven member.

25. The method of claim 24 wherein selectively engaging and disengaging at least one engagement member from the driven member comprises:
engaging the at least one engagement member with the driven member;
moving the striker against the energy storage means to the ejection position to store energy in the energy storage means;
engaging a locking means with at least a portion of the striker; and
releasing engagement of the at least one engagement member from the driven member.

26. The method of claim 25, wherein the ejecting comprises:
depressing a trigger to disengage an interlock; and
applying a distal portion of the needle-free injection device containing the at least one jet orifice to a desired injection site with a predetermined amount of pressure.
